(19) United States
(12) Patent Application Publication
(10) Pub. No.: US 2022/0022901 A1
(43) Pub. Date: Jan. 27, 2022

(54) FLOW PROTECTION DEVICE FOR ISCHEMIC STROKE TREATMENT

(71) Applicant: NeuroVasc Technologies, Inc., Irvine, CA (US)

(72) Inventors: Hyung Phouasalit, Costa Mesa, CA (US); Michael Losordo, San Juan Capistrano, CA (US)

(73) Assignee: NeuroVasc Technologies, Inc., Irvine, CA (US)

(21) Appl. No.: 17/496,119

(22) Filed: Oct. 7, 2021

Related U.S. Application Data
(63) Continuation of application No. 16/351,248, filed on Mar. 12, 2019.

(51) Int. Cl. A61B 17/221

(52) U.S. Cl. CPC A61B 17/221; A61B 2017/2215

(57) ABSTRACT

A clot removal device has an expandable treatment member, a proximal section, and a transition section having a distal end connected to the proximal end of the expandable treatment member, and a proximal end connected to the distal end of the proximal section. A delivery wire has a distal end coupled to the proximal section. The diameter of the proximal section is smaller than the diameter of the expandable treatment member, and the transition section has a diameter that varies from its proximal end to its distal end.

### BACKGROUND OF THE INVENTION

### 1. Field of the Invention

The present invention generally relates to devices and methods useful for clot retrieval, and removal devices to treat, among other things, ischemic stroke.

### 2. Description of the Prior Art

Currently, the FDA-approved treatment options for an acute ischemic stroke include intravenous (IV) delivery of clot dissolving medicine and mechanical thrombectomy.

For treatment use, clot dissolving medicine, such as the thrombolytic agent (Tissue Plasminogen Activator (t-PA)), is injected into the vasculature to dissolve blood clots that are blocking blood flow to the neurovasculature. Intravenous t-PA is currently limited in use because it must be used within a three-hour window from the onset of a stroke and can result in an increased risk of bleeding. This standard of care leaves room for upgrade, and is only the appropriate approach to treatment for a limited class of individuals, groups and temporally-limited exigent cases.

A second option includes the use of mechanical thrombectomy devices. Such devices are designed to physically capture an embolus or clot, and to remove it from the blocked vessel, thereby restoring blood flow. The major advantage of the mechanical thrombectomy device is it can expand the treatment window from three hours to over ten hours.

Some existing mechanical thrombectomy devices used for increasing blood flow through an obstructed blood vessel include: 1) a filter trap designed and built to collect and remove emboli; 2) a cork-screw guidewire-like device to retrieve embolus; and 3) a stent-like device connected to a delivery wire to retrieve embolus. All of these devices suffer from certain disadvantages.

First, filter-type thrombectomy devices tend to be cumbersome and difficult to deliver and deploy, and a larger-profile guide catheter may be needed to fully remove the embolus. In addition, it is difficult to coordinate precise and predictable movement to position the device properly in the vessel. The device can drift within the vessel, twist, or not be adequately conforming to the vessel wall and, therefore not effective for removing embolus.

Cork-screw guidewire devices can only capture and remove emboli that are firm, or subject to certain mechanical variables such as being held together by itself as one piece. Cork-screw guidewire devices are not effective in removing particulate matter that may be scattered or broken up.

Stent-like mechanical thrombectomy devices are not capable of capturing small emboli that break off from a large embolus (if any), and can lead to complications such as the blockage of distal smaller vessels, vessel dissection, perforation, and hemorrhage arising as a result of over-manipulation in the vessel.

The disadvantages common to all of the devices described above include, for example: 1) the device may capture an embolus, but then lose grasp of it and migrate/deposit it incidentally into another area of the neurovasculature, creating the potential for a new stroke in a different part of the neurovasculature; 2) the device is not capable of capturing small embolus breaking off from the larger embolus and preventing it from migrating to a more distal area of the neurovasculature; 3) the relative large device profile prevents these devices from treating the distal smaller diameter vessels; and 4) risk of sICH (symptomatic Intra-cerebral Hemorrhage) after intra-arterial clot removal in acute stroke patients.

Other flaws in the current mechanical thrombectomy designs include poor visibility/radiopacity, lack of variation in the delivery portion to enhance and improve deliverability, and lack of coatings or modified surface textures on the treatment portion to enhance embolus affinity, etc. In conclusion, there is a great need for improved devices, systems, and methods for restoring blood flow through a blood vessel. None of the existing medical mechanical thrombectomy devices address all necessary needs to date.

### SUMMARY OF THE DISCLOSURE

The present invention is directed to a method and devices for removing clots, emboli and other luminal blockages from a blood vessel. A clot removal device is provided, and has an expandable treatment member, a proximal section, and a transition section having a distal end connected to the proximal end of the expandable treatment member, and a proximal end connected to the distal end of the proximal section. A delivery wire has a distal end coupled to the proximal section. The diameter of the proximal section is smaller than the diameter of the expandable treatment member, and the transition section has a diameter that varies from its proximal end to its distal end.

The devices of the present invention can be made from either metallic biocompatible material (such as Nitinol, stainless steel, Co—Cr base alloy, Ta, Ti, etc.) or polymer based biocompatible material (polymers with shape memory effect, PTFE, HDPE, LDPE, Dacron, Polyester, etc.). For ischemic stroke treatment, the expandable treatment member must be flexible enough to negotiate the torturous vasculature of the brain and without modifying the vessel profile at the target location. The profile of the expandable treatment member must be small enough to reach target treatment site as known to artisans.

### BRIEF DESCRIPTION OF THE DRAWINGS

FIG. 1 is a side view of a fully expanded clot removal device according to a first embodiment of the present invention.

FIG. 2 is a side view of a fully expanded clot removal device according to a second embodiment of the present invention.

FIG. 3 is a side view of the clot removal device of FIG. 1 shown deployed for use inside a blood vessel.

FIG. 4 is a side view of an expandable treatment member of a fully expanded clot removal device according to a third embodiment of the present invention.

FIG. 5 is an enlarged view of the distal end of the expandable treatment member of FIG. 4.

FIG. 6 is a side view of a fully expanded clot removal device according to a fourth embodiment of the present invention.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention is directed to a device for removing emboli and other luminal blockages. The device includes an expandable treatment member, such as a mesh or a cage, and a proximal section that has a narrowed diameter in its expanded state. During treatment, the expandable treatment member is positioned proximal to an embolus within a blood vessel and then transitioned into an expanded state. In certain embodiments, the expandable treatment member's normal state is the expanded configuration, and the expandable treatment member is compacted and delivered to the treatment site in the compacted configuration through a delivery sheath or catheter. The expandable treatment member is deployed from the delivery sheath or catheter, which causes it to return to its normal expanded profile by the elastic energy stored in the device. Expansion of the expandable treatment member creates a cylindrical space in the vessel in a location just proximal to the emboli/clots. A proximal section with a smaller diameter compared to the expandable treatment member has overlap with the delivery catheter (partially or entirely within the delivery catheter) to create the lumen/channel for aspiration through the catheter. The transition section is located between the expandable treatment member and the proximal section. The provision of the transition section combined with the expandable treatment member advantageously limits or restricts forward blood flow and creates a pressure gradient within the blood vessel between locations distal and proximal to the device. The pressure gradient helps to prevent the clots from being flushed away from the treatment member, thereby assisting in removal of the embolus from the blood vessel. Specifically, the pressure difference through aspiration can act like a vacuum to assist in removal of the embolus from the blood vessel. Under aspiration, the emboli and clots can be pulled inside the expandable treatment member, then the expandable treatment member and the emboli engaged with the expandable treatment member are removed from the blood vessel. During clot removal, the expandable treatment member (with the blood clot engaged) can also be pulled inside a guiding or delivery catheter, and removed from the blood vessel. Furthermore, aspiration/vacuum suction can be applied through the lumen of the access catheter lumen and the proximal section to prevent clots from breaking off and flowing downstream.

In addition, the transition section regulates the forward blood flow and allows the controlled (gradual) restoration of the blood flow, and reduces the risk of sICH (symptomatic Intra-cerebral Hemorrhage) after intra-arterial clot removal in acute stroke patients.

Devices of the present invention are suitable for removal of blockages in body lumens, and are particularly well-suited for removal of thrombi, emboli, or atheroma in the vasculature, including those in arteries and veins. It is understood that the dimensions of the device may be modified to suit a particular application. For example, devices of the invention used for treatment of deep vein thrombosis may have a larger cross-section than devices of the invention used for treatment of brain ischemic.

Compared with existing mechanical thrombectomy devices, the unique device design provided by the present invention has the advantage of providing a proximal flow restriction feature to block the forward flow of blood when the device is deployed during use. This feature can help to eliminate or reduce the risk of flush, or the break-up of the blood clots during the procedure aid to a faster and complete clot removal.

Another important advantage provided by the present invention is the central lumen of the proximal section can be used or combined with the lumen of the access catheter to apply aspiration/suction force to help with the complete removal of the blood clots in the vasculature.

Thus, the device described in the present invention overcomes the shortcomings of the existing technologies and can be delivered to the target vasculature smoothly, can be retrieved safely, and can remove the entire embolus with fewer passes. In use, the mechanical thrombectomy device described in the present invention can be compacted to a low profile and loaded onto a delivery system and delivered to the target location in the vessel by a Medical procedure such as through use of a delivery catheter. The mechanical thrombectomy device can be released from the delivery system when it reaches the target implant site and expanded to its normal expanded profile by the elastic energy stored in the device (self-expandable device).

As for the relative position of the expandable treatment member in relation to the embolus or blood clot, it can be deployed at a site proximal to the embolus. In dealing with long embolus, the expandable treatment member can also be used to remove the embolus from the proximal portion to the distal portion with multiple passes, until the entire embolus is removed.

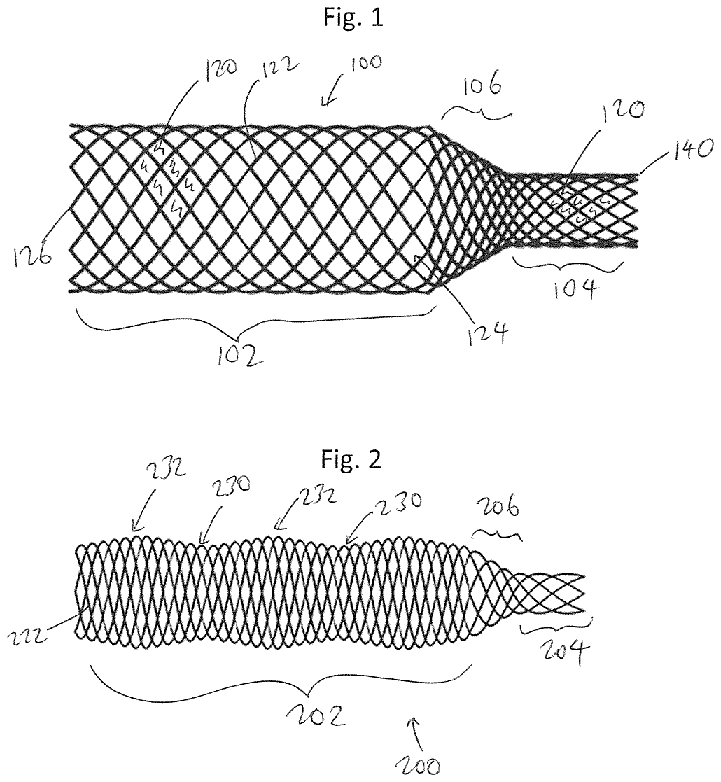

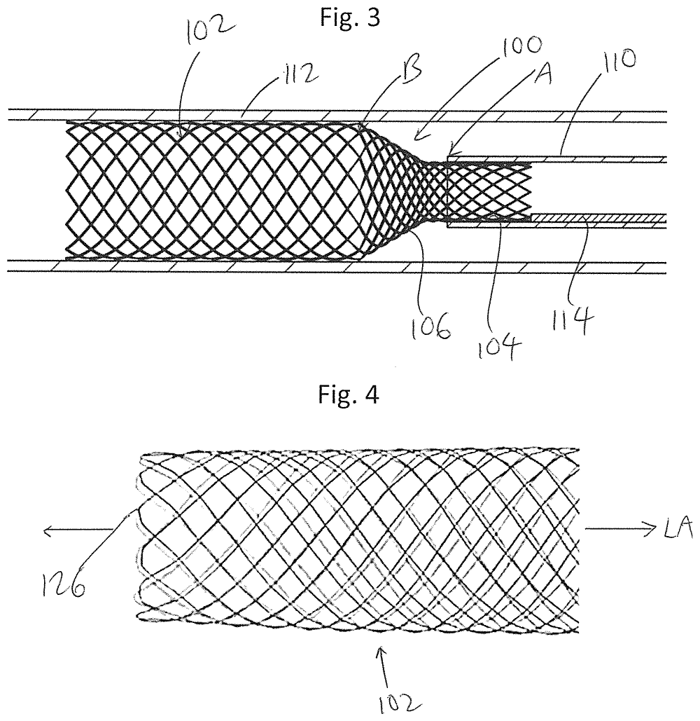

FIGS. 1 and 3 illustrate a device 100 for removing emboli and other luminal blockages according to the present invention. The device 100 can be made from one piece or multiple pieces of Nitinol™ super elastic material or Nitinol™ super-elastic alloy tubing. It can also be made from other biocompatible materials that exhibit super-elastic or shape memory properties. The device 100 can be made by laser cutting, mechanical machining, chemical machining, electrochemical machining, EDM, braiding and related techniques known to those skilled in the art.

The device 100 has an expandable treatment member 102, a proximal section 104 and a transition section 106. The proximal end 140 of the proximal section 104 defines an open mouth or open end for the lumen of the device 100, while the distal end 126 of the expandable treatment member 102 defines an open mouth or open end for the lumen of the device 100. The expandable treatment member 102 and the proximal section 104 can have different diameters in their expanded states, with the expandable treatment member 102 having a larger diameter than the proximal member 104. The transition section 106 can be a tapered section that tapers from the expandable treatment member 102 to the proximal section 104. The taper for the transition section 106 can be a continuous taper, or it can stepped (not shown).

The device 100 can be comprised of a braided mesh or laser cut element. The device 100 can be attached to a delivery wire 114 and can be introduced into a body lumen 112 via a catheter 110. The device 100 can expand to its expanded diameter when released from the catheter 110, with the expandable treatment member 102 expanding to a diameter of 2 mm to 10 mm.

The braided mesh or laser cut element may have a variable thickness along the length of the device 100 to enable easier compression and delivery of the device 100, and to reduce the overall bulk size at the proximal attachment region between the braided mesh or laser cut element and the delivery catheter 110.

The transition section 106 may contain a single or multiple transition sections between the multiple sections. These transition sections are also self-expanding and can be designed to function as a barrier when deployed at the mouth (open distal end) of a catheter 100 or in a vessel 112, as the transition section 106 can create a seal from the mouth of the catheter 110 to the wall of the vessel 112.

Each of the expandable treatment member 102 and the proximal section 104 may be a constant diameter or a variable cylindrical or oval shape, such as repeating between smaller and larger diameters. An example is shown in the embodiment of FIG. 2 described below. This variable profile can provide better conformability in a vessel with an inconsistent diameter due to calcification or other diseased states.

The proximal section 104 may be designed to conform to the inner diameter/surface of a catheter 110, and the distal expandable treatment member 102 may be designed to conform to the inner diameter of a vessel 112 when released from a catheter 110.

The proximal end of the proximal section 104 may be attached to a delivery element (e.g., the delivery wire 114) along the outer diameter (i.e., the side of the structure) of the proximal section 102, thereby enabling a maximum lumen size for aspiration through the lumen. The delivery element (e.g., delivery wire 114) is not attached along the central axis of the lumen of the catheter 110 or the proximal section 104.

The inner lumen of the device 100 may be open to enable other devices or the clot to be pushed or pulled through without obstruction, and to achieve maximum aspiration through it.

The device 100 may be coated in full or in part with a covering or coating 120, with a single or multiple layers of different coating or covering materials. For example, in one embodiment, the transition section 106 and the proximal section 104 can be left uncovered, while the distal expandable treatment member 102 (which has a larger diameter) is covered. The surfaces of the proximal section 104 and the transition section 106 can be either completely uncovered, or entirely or partially covered, by the coating 120. The coating 120 can be a polymer material that functions to restrict the blood flow.

The coating 120 can be applied on the internal surface of the device, or the outer surface of the device 100. The coating 120 can also be applied onto both the internal and outer surfaces of the device 100. The coating 120 can provide a variable porosity for the device, as well as increased lubricity. The coating 120 can also be used to totally or partially cut off the blood communication in the vessel 112.

The entire device 100 can be collapsed into a compressed state having a diameter of 0.010 inches to 0.50 inches, or less, to enable delivery through a catheter 110. The device 100 can be made from Nitinol™ or a combination of other superelastic materials and radiopaque materials. The device 100 may contain radiopaque markers in the form of wires, coils or tubular pieces (such as marker band, etc.).

The device 100 may be used with a guiding or intermediate catheter to regulate flow in a vessel 112, and to pull clots, thrombi, or other emboli into the catheter 110 in conjunction with aspiration.

The device 100 can be a meshed frame throughout, and the meshed frame can be provided with a plurality of openings 124. Frame members or struts 122 form the body of the meshed frame and define the plurality of openings 124. In certain embodiments, the struts 122 are a plurality of intersecting wires or other threads. The struts 122 may form a mesh or cage-like structure that defines the plurality of openings 124.

In certain embodiments, the expandable treatment member 102 can include a plurality of protrusions (not shown) on the frame. The plurality of protrusions further engages the embolus for removal. As an alternative to, or in addition to, the plurality of protrusions, the expandable treatment member 102 may include one or more surface modifications or treatments, as described below. For example, the surface of the expandable treatment member 102 may be roughened to improve clot adhesion. The longitudinal axis of the expandable treatment member 102 can also be offset or different from the longitudinal center axis of the native blood vessel. When the expandable treatment member 102 is in use, both the delivery catheter (e.g., catheter 110) and/or the movement axis of the expandable treatment member 102 can be different from the longitudinal central axis of the vessel 112, and can contact the side wall of the blood vessel 112.

The delivery wire 114 can be made of super-elastic Nitinol wire, stainless steel wire, braided stainless steel wire, Co—Cr alloy and other biocompatible materials. The diameter of the delivery wire 114 can range from 0.008″ to 0.030″, and the delivery wire 104 can have variable diameters/stiffness along its length.

This distal end 126 of the expandable treatment member 102 can have markers made from Ta, Pt, W, Pt—W, or Pt—Ir alloys for radiopacity, and from radiopaque coils or markers.

The proximal section 104 can be fabricated from the one or two element(s) of the device 100, or fabricated from other pieces of material, then attached to the delivery wire 114 by mechanical means, or via a thermal (laser or soldering) process, or adhesive/glue, or heat shrink technology.

The diameter of the proximal section 104 can range from 0.5 mm to 12 mm, and its length can range from 2 mm to 100 mm.

The diameter of the transition section 106 can range from to 2 mm to 10 mm at the distal end of the proximal section 104, to 2 mm to 10 mm at the proximal end of the expandable treatment member 102, and its length can range from 1 mm to 10 mm.

The diameter of the expandable treatment member 102 can range from 2 mm to 10 mm, and its length can range from 5 mm to 60 mm.

Radiopaque markers can be attached on any portion of the device 100 for positioning. One way to provide full visibility for the device 100 is to run a radiopaque material through the entire or partial lumen of the delivery wire 114. Markers can also be placed on the expandable treatment member 102 to aid in positioning. In addition, radiopaque markers (marker coils, marker bands, radiopaque wire(s), radiopaque coatings, etc.) can be integrated into the proximal section 104.

The device 100 can be made entirely from a braided wire, and some radiopaque wires can be integrated into the braid for better radiopacity. The angles of the braided wire mesh may vary along the entire length thereof.

The device 100 can have a surface treatment on selected portions to improve performance for the selected portions of the device 100. Both the proximal section 104 and the expandable treatment member 102 can either be coated or covered, entirely or partially, by typical biocompatible materials for lubricity. The surface of the expandable treatment member 102 can have either a positive or negative charge for improved clot adhesion. The surface of the expandable treatment member 102 can also be either mechanically or chemically treated to have a “rough” surface for improved clot adhesion. The “rough” surface can be achieved by (i) a porous surface coating or layer (ii) a micro blasted surface or micropinning, or (iii) an irregular strut geometry or arrangement.

The expandable treatment member 102 can be fully or partially coated with chemical(s), drug(s) or other bioagents to prevent clotting and/or for the better adhesion between the device and embolus. In addition, the surfaces of the expandable treatment member 102 and the proximal section 104 can be treated to form different surface layers (e.g., oxidation layer, Nitro or carbonized or N—C-combined surface layer, etc.) for better adhesion between the expandable treatment member 102 and the embolus.

In use, a guide wire can be inserted through the vasculature to the target treatment site, and then the catheter 110 is delivered over the guide wire to a target location in a vessel with the device 100 housed therein using conventional delivery techniques that are known to those skilled in the art. Alternatively, the catheter 110 can be inserted over the guide wire first, then the compacted device 100 can be inserted through the inner lumen of the catheter 110. The distal end of the catheter 110 can be positioned proximal to the clot or embolus at the target location, and there is no need for the catheter 110 to traverse the clot or embolus, thereby minimizing the possibility of pushing the clot or embolus downstream in the vessel.

The catheter 110 can then be pulled back (proximally) to expose first the expandable treatment member 102, then the transition section 106, and then a portion of the distal portion of the proximal section 104, as shown in FIG. 3. Instead of pulling back the catheter 110, it is also possible to deploy the expandable treatment member 102 by inserting the device 100 into the catheter 110 until the distal end 126 reaches the distal end of the catheter 110, and then holding the proximal end of the catheter 110 in a stationary position, pushing the device 100 distally out of the catheter 110. Under this alternative, there is no need to withdraw the catheter 110, which allows the positioning to be more accurate. The expandable treatment member 102 will then fully deploy (i.e., reach its largest diameter) to create a cylindrical space proximal to the clot to aid in aspiration and removal of the clot. At this point, the catheter 110 and the elongated delivery wire 114 will be pulled back or withdrawn at the same time to remove the clot.

During this procedure, the device 100 apposes the distal mouth of the catheter 110 to form a seal at the location A in FIG. 3. In addition, the device 100 apposes the vessel wall 112 to restrict flow to achieve partial or complete flow restriction to minimize the risk of poor clot retention and clot dislodgement. The expandable treatment member 102 can collect all the clots/emboli inside the cylindrical space to prevent them from flowing downstream. Adjusting the position of the proximal section 104 also regulates the flow of blood during and immediately after the procedure to eliminate the effect of sICH for a better clinical outcome.

In addition, aspiration or suction can be applied from the proximal end of the catheter 110 to pull smaller clots and particles into the proximal section 104 using suction force, and then removed from the blood vessel 112. The suction/aspiration action through the lumen of the access devices (e.g., the catheter 110) and the encapsulation of the expandable treatment member 102 (with clot engaged) can happen either simultaneously or in sequence during the procedure.

The description herein discloses a technique when the device 100 is used alone as an aspiration device for clot removal. In addition, the device 100 can also be used along with other conventional mechanical thrombectomy devices (such as the Solitaire™ device from Medtronic, and the Trevo™ device from Stryker, among others) to improve the removability of the clot. When combined with other mechanical thrombectomy devices, the device 100 can be deployed in a more distal location in the vessel 112, typically on the clot or distal to the clot, and then the expandable treatment member 102 of the device 100 is deployed proximal to the other conventional mechanical thrombectomy device. The transition section 106 can regulate the forward blood flow, while the aspiration can be applied through the lumen of the proximal section 104 and the catheter 110. The conventional mechanical thrombectomy device with the clot engaged can then be pulled inside the expandable treatment member 102, and the entire system can be removed from the vessel 112.

FIG. 2 illustrates another embodiment of a device 200 under the present invention. The device 200 is the same as the device 100 and has an expandable treatment member 202, a proximal section 204 and a transition section 206. However, in the device 200, each of the expandable treatment member 202, the transition section 206 and the proximal section 204 can have varying diameters in their expanded states, but the smallest diameter of the expandable treatment member 202 will be larger than the largest diameter of the proximal member 204. In addition, the transition section 206 can be a tapered section that tapers from the expandable treatment member 202 to the proximal section 204. The taper for the transition section 206 can be a continuous taper, or it can stepped (not shown). The smallest diameter at the proximal end of the transition section 206 should be the same as, or larger than, the largest diameter of the proximal section 204.

As shown in FIG. 2, the longitudinal length of the expandable treatment member 202 has an undulating wall which has smaller diameter sections 230 and larger diameter sections 232, thereby providing a variable outer contour. The pattern and arrangement of these varying diameter sections can be consistent or irregular, and can depend on the vasculature for which the device 200 is used. Preferably, the undulations are curved and smooth so as to minimize trauma to the vessel wall. In addition, the coatings, surface treatments and markers described above can also be provided to the device 200.

In addition, the expandable treatment member 202 can be provided with struts 222 that have a greater thickness than the thickness of the struts 222 at the proximal section 204. The struts 222 in the transition region 206 can have a thickness that is the same as the thickness of either the expandable treatment member 202, the proximal section 204, or can have a thickness that is different from the thicknesses of the expandable treatment member 202 and the proximal section 204. For example, the thickness of the struts 222 in the transition region 206 can be smaller than the thickness of the struts 222 in the expandable treatment member 202, but greater than the thickness of the struts 222 in the proximal section 204. In addition, the thickness of the struts 222 in the transition section 206 can even be varied from its proximal end to its distal end. In fact, the thickness of the struts 222 in the expandable treatment member 202 can even be varied along its length in any consistent or random manner, again depending on the clinical use.

The distal end 126 of the expandable treatment member 102 may be comprised of individually terminating struts 122 or may be comprised of closed-end or rounded struts for improved strut arrangement when compressed, so as to facilitate easier delivery. FIGS. 4-6 illustrate various embodiments of these distal end 126 strut configurations.

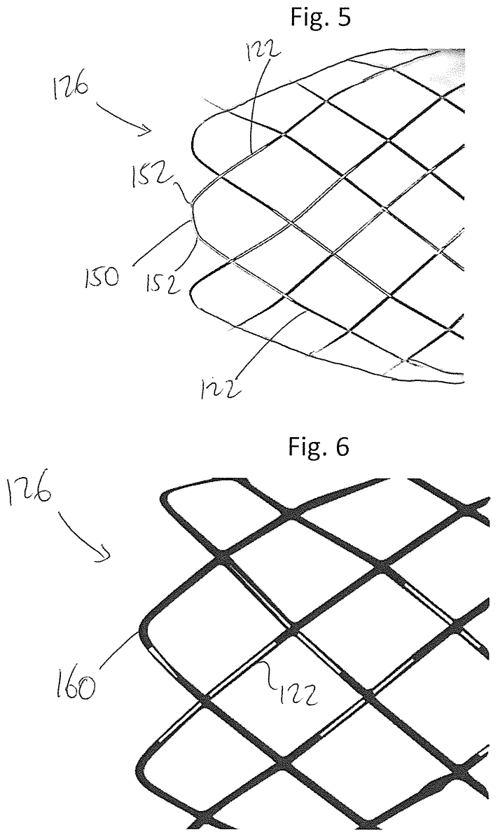

FIGS. 4-5 illustrate the distal end 126 of a expandable treatment member 102 where discrete linear segments 150 are provided when in a closed-end configuration, with the linear segment 150 being perpendicular to the longitudinal axis LA of the device 100. The linear segments 150 extend from struts 122 at distinct bend points 152 such that during compression of the device 100, the struts 122 bend at these points. The linear segment 150 can have a length of about 0.1 mm to 10 mm.

FIG. 6 illustrates the distal end 126 of another expandable treatment member 102 where the struts 122 meet at rounded or curved nodes 160. These rounded nodes 160 may be rounded to have a radius that is about 0.01 mm to 5 mm.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A clot removal device, comprising:
an expandable treatment member having a diameter, a distal end and a proximal end;
a proximal section having a diameter, a distal end and a proximal end;
a transition section having a distal end connected to the proximal end of the expandable treatment member, and a proximal end connected to the distal end of the proximal section;
a delivery wire having a distal end coupled to the proximal section; and
wherein the diameter of the proximal section is smaller than the diameter of the expandable treatment member, and wherein the transition section has a diameter that varies from its proximal end to its distal end.

2. The device of claim 1, wherein the varying diameter of the transition section tapers from a smaller diameter at its proximal end to a larger diameter at its distal end.

3. The device of claim 1, further including a coating provided on the proximal section.

4. The device of claim 1, further including a coating provided on the transition section.

5. The device of claim 3, further including a coating provided on the transition section.

6. The device of claim 1, wherein the expandable treatment member has a varying contour.

7. The device of claim 1, wherein the distal end of the expandable treatment member has lateral segments.

8. The device of claim 1, wherein the distal end of the expandable treatment member has rounded nodes.
